(19) United States
(12) Patent Application Publication
(10) Pub. No.: US 2015/0250521 A1
(43) Pub. Date: Sep. 10, 2015

(54) Driver Apparatus For A Pedicle Screw Assembly

(71) Applicant: DEROYAL INDUSTRIES INC., Powell, TN (US)

(72) Inventors: Nicholas Poker, Knoxville, TN (US); Travis Lane Turley, Knoxville, TN (US); Nephi Zufelt, Knoxville, TN (US)

(21) Appl. No.: 14/432,782

(22) PCT Filed: Oct. 15, 2013
(86) PCT No.: PCT/US13/65074

§ 371 Date: Apr. 1, 2015

Related U.S. Application Data
(60) Provisional application No. 61/715,612, filed on Oct. 18, 2012.

(51) Int. Cl. A61B 17/88

(52) U.S. Cl. CPC A61B 17/8875

(57) ABSTRACT

A driver apparatus for a pedicle screw assembly includes a release shaft rotatably mounted in a driver housing. A lower end of the driver housing is configured to be received in a slot in the upper end of a connector housing of a pedicle screw assembly. The release shaft has a thread on its lower end which can be made up or unmade from threaded engagement with the internal threads of the connector housing of the pedicle screw assembly so as to connect or disconnect the driver apparatus from the pedicle screw assembly.

### TECHNICAL FIELD

The present invention relates generally to a driver apparatus for a pedicle screw assembly for providing an anchor point to a spinal segment for spinal fusion surgery.

### DISCLOSURE OF THE INVENTION

In one embodiment a driver apparatus for a pedicle screw assembly is provided. The pedicle screw assembly includes a pedicle screw and a connector housing mounted on the pedicle screw. The driver apparatus includes an elongated driver housing having a proximal housing end and a distal housing end. The distal housing end includes a screw drive head configured for engaging the pedicle screw so that the pedicle screw can be rotated by rotating the driver housing. The driver housing has a first laterally open window located adjacent the proximal housing end, and has a second laterally open window located adjacent the distal housing end. The driver housing has a longitudinal housing passage defined therein communicating the first and second laterally open windows. A release shaft extends through the longitudinal housing passage. The shaft has a knob attached to a proximal end portion of the shaft and has a release thread defined on the distal end portion of the shaft. The knob is received in and accessible through the first laterally open window. The release thread is received in and accessible through the second laterally open window. The release shaft is rotatable within the driver housing. The driver housing includes a guide surface extending from the second laterally open window distally toward the screw drive head. The guide surface is configured to hold the connector housing of the pedicle screw assembly against rotation relative to the driver housing when the release shaft is rotated relative to the driver housing to disconnect the release thread from the connector housing of the pedicle screw assembly.

In another embodiment a pedicle screw installation system includes a pedicle screw assembly and a driver apparatus. The pedicle screw assembly includes a pedicle screw and a connector housing mounted on the pedicle screw. The pedicle screw includes an enlarged ball end. The connector housing has a lower seat receiving the ball end with the screw extending downward through a lower opening of the connector housing. The connector housing has two upwardly extending arms having internal threads defined on the arms. The connector housing has a slot dividing the two arms. The driver apparatus includes an elongated driver housing including a screw drive head defined on a lower end of the driver housing. The screw drive head is configured for engaging the ball end of the pedicle screw so that the pedicle screw can be rotated by rotating the driver housing. The driver housing has an upper laterally open window and a lower laterally open window. The driver housing has a longitudinal passage defined therein communicating the upper and lower laterally open windows. The driver housing includes first and second longitudinally extending guide surfaces on opposite sides of the lower laterally open window. The guide surfaces are configured to be received in the slot of the connector housing so that the connector housing may move longitudinally but not rotationally relative to the driver housing when the guide surfaces are received in the slot of the connector housing. The driver apparatus further includes a release shaft extending through the longitudinal housing passage. The shaft has a knob attached to the shaft. The knob is received in and accessible through the upper laterally open window. The shaft has a release thread defined on the shaft and extending laterally through the lower laterally open window. The release thread is configured to be threadedly engaged with the internal threads of the two arms of the connector housing. The release shaft is rotatable within the driver housing to selectively engage or disengage the release threads from the internal threads of the arms of the connector housing to connect or disconnect the driver from the pedicle screw assembly.

In another embodiment a method of installing a pedicle screw assembly in a patient comprises:

(a) providing a driver having a driver housing and having a release shaft rotatably received in the driver housing;

(b) providing the pedicle screw assembly including a pedicle screw and a connector housing, the connector housing having two upwardly extending arms with a slot between the arms;

(c) inserting a guide portion of a lower end of the driver housing into the slot of the connector housing;

(d) making up a threaded engagement of a release thread of the release shaft with internal threads of the upwardly extending arms of the connector housing;

(e) during step (d), advancing the guide portion of the lower end of the driver housing into the slot while preventing rotation of the connector housing relative to the driver housing, and forcing a drive head on a lower end of the driver housing into engagement with the pedicle screw; and

(f) substantially circumferentially enclosing the release threads within the arms of the connector housing and the guide portion of the driver housing.

In any of the above embodiments the driver apparatus may further include an elongated spool handle rotatably received about the driver housing.

In any of the above embodiments the first laterally open window may be located in an enlarged diameter portion of the driver housing.

In any of the above embodiments the knob may protrude through the first laterally open window on two diametrically opposed sides of the driver housing so that the knob can be grasped between the thumb and forefingers of the human hand.

In any of the above embodiments the driver housing may comprise a multi-part housing assembly including a tubular central housing portion having a proximal end and a distal end. An enlarged diameter knob housing portion may be attached to the proximal end of the central housing portion and has the first laterally open window defined therein. A distal housing portion may be attached to the distal end of the central housing portion and has the second laterally open window defined therein.

In any of the above embodiments the guide surface may comprise first and second diametrically opposed parallel guide surface portions on opposite sides of the lower laterally open window.

In any of the above embodiments the axially overlapping portions of the driver housing and the upward extending arms of the connector housing may substantially enclose the release threads to shield the release threads from engagement with a patient's body tissue as the pedicle screw is inserted into the patient's body.

In any of the above embodiments, the engagement of the driver apparatus with the pedicle screw assembly may place a tension load across the connector housing between the pedicle screw and the internal threads of the arms of the connector housing, thereby holding the pedicle screw and the connector housing in axial alignment with the driver so as to resist misalignment of the pedicle screw and the connector housing during the application of driving torque to the pedicle screw by the driver.

Numerous objects features and advantages of the present invention will be readily apparent to those skilled in the art upon a reading of the following disclosure when taken in conjunction with the accompanying drawings.

### BRIEF DESCRIPTION OF THE DRAWINGS

FIG. 1 is a schematic cross-section view showing two pedicle screw assemblies in place within a lumbar vertebra and showing two elongated rod members fixed in place within connector housings of the pedicle screw assemblies.

FIG. 2 is an exploded view of one of the pedicle screw assemblies.

FIG. 3 is a perspective view of an upper housing portion of the pedicle screw assembly.

FIG. 4 is a cross-sectional view of the upper housing portion of FIG. 3.

FIG. 5 is a perspective view of a sleeve of the pedicle screw assembly.

FIG. 6 is a cross-sectional view of the sleeve of FIG. 5.

FIG. 7 is a perspective view of a lower housing portion of the pedicle screw assembly.

FIG. 8 is a cross-sectional elevation view of the lower housing portion of FIG. 7.

FIG. 9 is a perspective view of an upper nut of the pedicle screw assembly.

FIG. 10 is a cross-section elevation view of the upper nut of FIG. 9.

FIG. 11 is a plan view of an annular wave spring of the pedicle screw assembly.

FIG. 12 is a schematic cross-section view of the wave spring of FIG. 11.

FIG. 13 is a perspective view of a driver apparatus for the pedicle screw assembly of FIG. 2, showing one of the pedicle screw assemblies in place on the distal end of the driver apparatus.

FIG. 14 is an elevation view of an upper driver housing portion.

FIG. 15 is an elevation partly sectioned view of a thumb wheel of the driver apparatus of FIG. 13.

FIG. 16 is an elevation view of a lower driver housing portion.

FIG. 17 is a cross-sectional elevation view of the lower driver housing portion of FIG. 16.

FIG. 18 is an elevation sectioned view of a tubular central driver housing portion.

FIG. 19 is an elevation sectioned view of an elongated spool handle of the driver apparatus.

FIG. 20 is an elevation view of a release shaft of the driver apparatus.

FIG. 21 is an elevation view showing the lower end of the driver apparatus about to engage with a pedicle screw assembly.

FIG. 22 is an elevation cross-sectioned view showing the lower end of the driver apparatus fully engaged with the pedicle screw assembly.

FIG. 23 is an elevation view showing the lower end of the driver apparatus connected to the pedicle screw assembly.

FIG. 24 is a right side elevation view of the apparatus of FIG. 23.

### BEST MODE FOR CARRYING OUT THE INVENTION

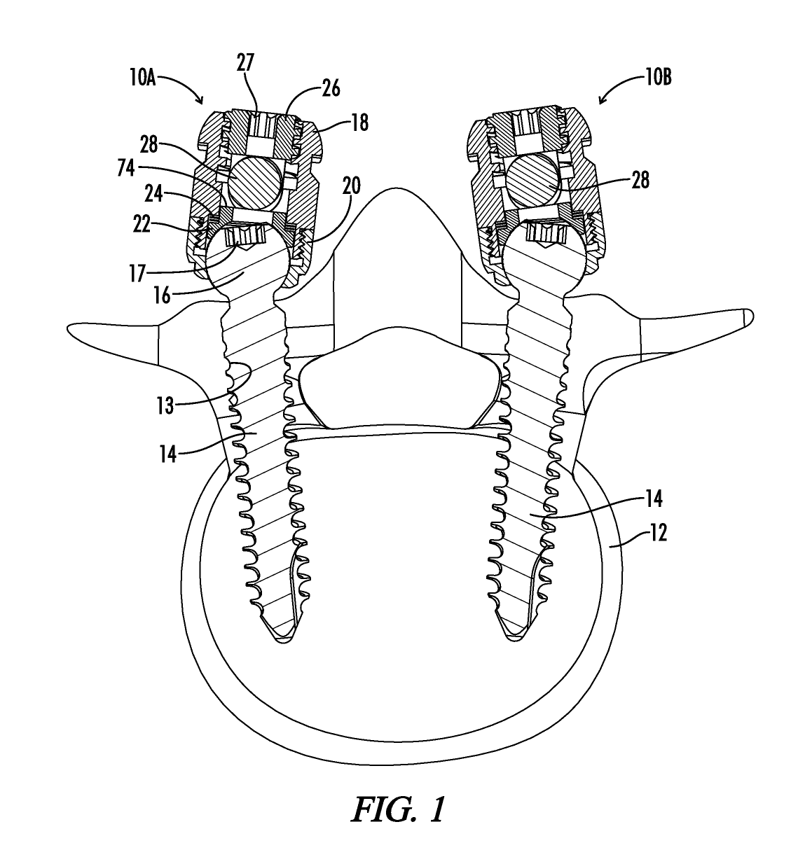

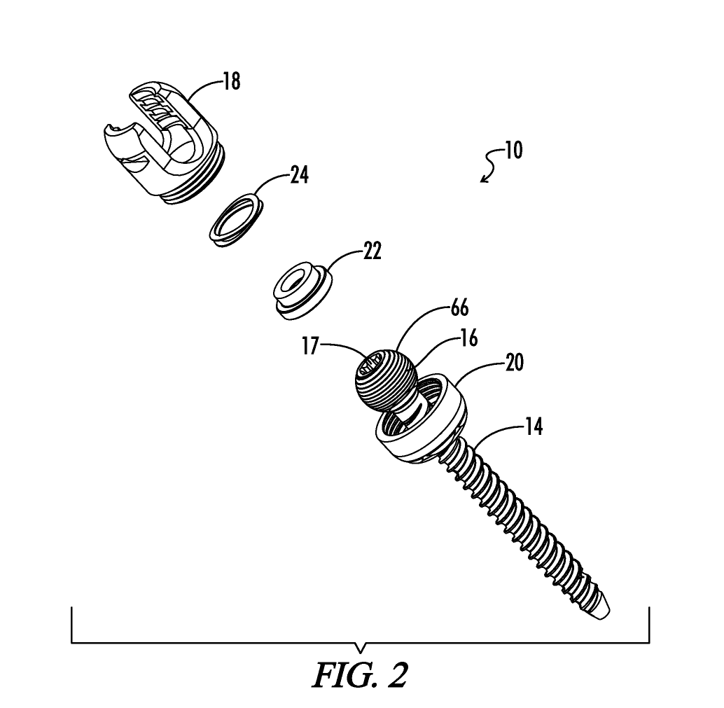

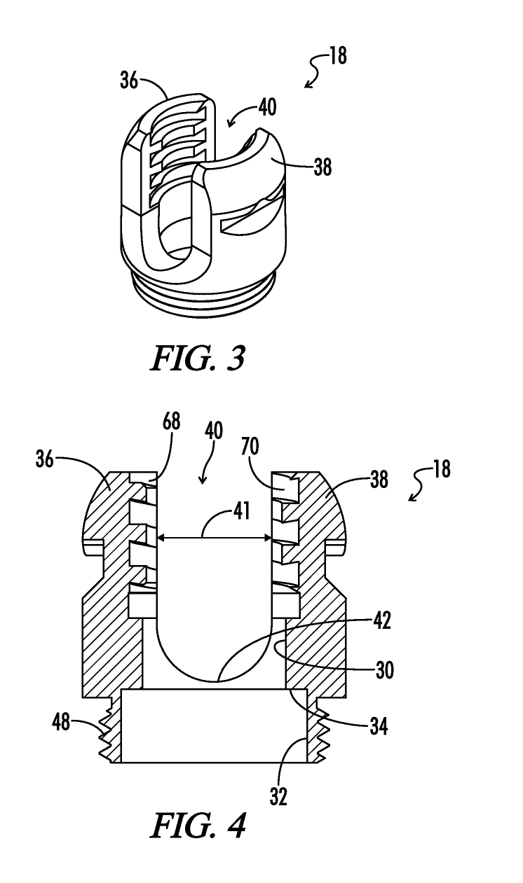

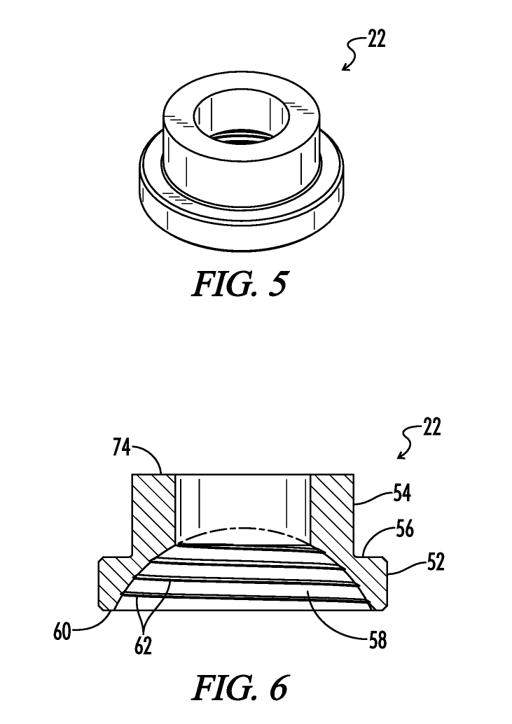

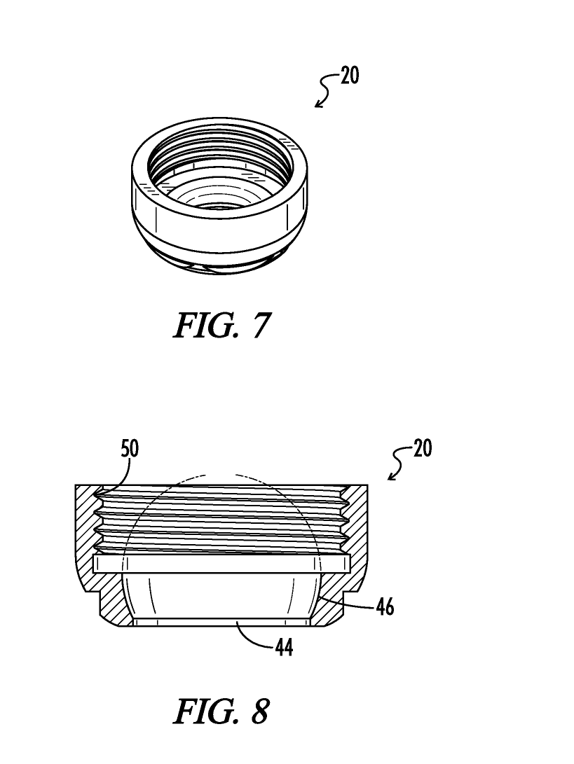

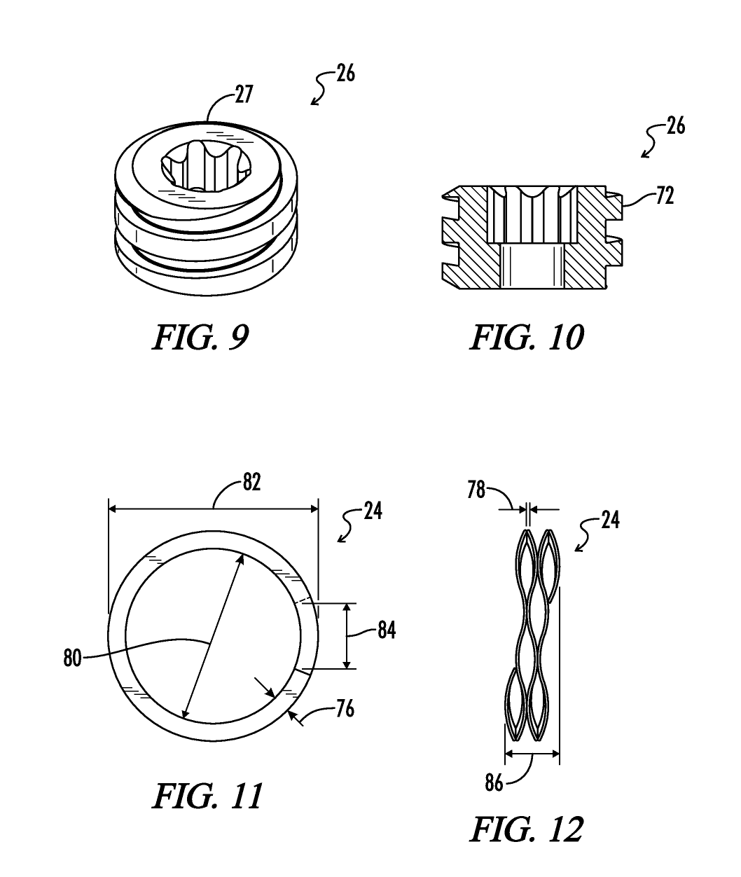

Referring now to FIGS. 1 and 2, a pedicle screw assembly is generally shown in exploded view in FIG. 2 and designated by the numeral 10. In FIG. 1 two of the pedicle screw assemblies are shown in place within a spinal segment 12, and the two pedicle screw assemblies are designated 10A and 10B.

Each of the pedicle screw assemblies 10 includes an elongated screw 14 having an enlarged ball end 16, an upper housing part 18 and a lower housing part 20, a sleeve 22, an annular wave spring 24, and an upper nut 26. The housing 18, 20 is sometimes referred to as a tulip housing or connector housing.

The enlarged ball end 16 has a multi-lobed socket 17 formed in the upper end thereof for engagement with a drive head of the driver apparatus 100 as further described below.

As seen in FIG. 1, each of the pedicle screw assemblies 10 is utilized to fix an elongated rod or elongated member 28 in place relative to the vertebra 12.

As best seen in FIG. 4, the upper housing portion 18, which may be also referred to as an upper housing part 18, has an axial bore 30 therethrough and has a lower counterbore 32. A downward facing annular surface 34 joins the bore 30 and the counterbore 32. The upper housing part 18 has two upward or longitudinally extending arms 36 and 38 separated by a central slot 40 for receiving the elongated rod 28 transversely through the slot 40. The slot 40 has a bottom 42.

The lower housing part 20 seen in FIG. 8 has a lower opening 44 through which the screw 14 is received. The lower housing part 20 has a lower seat 46 located above the lower opening 44. The lower seat 46 is shaped complementary to the ball end 16 for receiving the ball end 16 as seen in FIG. 1.

The lower end of upper housing part 18 has an external thread 48 which is received within an internal thread 50 of the upper end of lower housing part 20 so that the upper housing part 18 and the lower housing part 20 may be threadedly connected together by threads 48, 50.

The sleeve 22 has a lower larger diameter end portion 52 received in the counterbore 32 of the upper housing part 18. The sleeve 22 further has an upper smaller diameter end portion 54 which is received in the bore 30 of the upper housing part 18 as seen in FIG. 1. The sleeve 22 has an upward facing annular shelf 56 joining the larger and smaller diameter portions 52 and 54. The sleeve has an upper seat 58 defined on a lower end 60 of the sleeve 22. The upper seat 58 is shaped complementary to the ball end 16 for receiving the ball end 16 therein as seen in FIG. 1.

The annular wave spring 24 is disposed concentrically about the upper smaller diameter end portion 54 of the sleeve 22 and as seen in FIG. 1 is sandwiched between the downward facing annular surface 34 of the upper housing part 18 and the upward facing annular shelf 56 of the sleeve 22. The sleeve 22 is axially slidable relative to the upper housing part 38 and the annular wave spring 24 is dimensioned so as to provide a frictional pre-load on the ball end 16 sandwiched between the upper and lower seats 58 and 46 prior to fixation of the elongated rod 28 in the upper housing part 18.

The upper seat 58 may have scribe lines or grooves 62 therein. The ball end 16 may have scribe lines or grooves 66 defined therein. The use of the scribe lines or grooves 62 and 66, in any desired combination, increases friction between the seats and the ball end.

The upward extending arms 36 and 38 of the upper housing part 18 have internal threads 68 and 70 defined thereon. The upper nut 26 has a matching external thread 72 for receipt within the internal threads 68 and 70. The threads 68, 70 and 72 may for example be in the form of a single sided dove-tail threadform.

As best seen in FIG. 1, when the pedicle screws are in place and it is desired to fix the elongated rod 28 relative to the pedicle screw, the upper nut 26 is threadedly engaged with the threads 68 and 70 of the arms 36 and 38 to fix the elongated rod in the upper housing part 18. As can be seen in FIG. 1, an upper end 74 of the sleeve 22 extends above the bottom 42 of the slot 40 so that when the upper nut 26 is securely engaged with the elongated rod 28 for fixation of the elongated rod 28 in the upper housing part 18, a clamping force is applied by the upper nut through the elongated rod 28 to the sleeve 22 to also clamp the ball end 16 between the upper and lower seats 58 and 46. This clamping force is transmitted without passing through the annular wave spring 24.

The purpose of the annular wave spring 24 is to provide a pre-load on the sleeve 22 thus providing a pre-load sandwiching the ball end 16 between the upper and lower seats 58 and 46. This provides a frictional resistance of the housing assembly 18, 20 relative to the pedicle screw 14. The surgeon may rotate the housing assembly to a desired orientation relative to the pedicle screw 14 and the housing assembly will generally stay in place as the elongated rod 28 is placed therethrough. This allows the surgeon to assemble the spinal reinforcing structure made up of a plurality of the pedicle screw assemblies 10 and a plurality of the elongated rods 28 and associated cross-pieces (not shown).

The construction of the annular wave spring 24 is best seen in FIGS. 11 and 12. The wave spring illustrated is of the type generally referred to as a crest-to-crest wave spring, meaning that the crests of the waves as seen for example in FIG. 12 are joined together between adjacent coils of the wave spring. Other non crest-to-crest wave spring types may also be used. The spring for example may be constructed from a flat titanium wire having a width 76 and a thickness 78. The spring 24 may have an inside diameter 80 and an outside diameter 82, the difference in which is equal to the wire width 76.

In one embodiment, the spring 24 may comprise slightly less than two complete coils, with the coil ends being separated by a gap 84. This can be described as comprising at least 1-½ complete coils. In one example, such a spring may be made from grade 2 titanium wire having a wire width 76 of 0.81 mm and a wire thickness 78 of 0.25 mm. The spring may have an outside diameter 82 slightly less than 9 mm in order to fit within a 9 mm bore. The wave pattern may provide 3-1/2 waves for each 360° coil of the spring. The spring may have a free height 86 as seen in FIG. 12 of 1.30 mm. Other spring types having less than one and on-half coils may also be used. For example, a single coil wave spring may be used.

An annular wave spring 24 of the type just described provides a uniform application of force around the ball end 16 thus providing for smooth and reliable operation of the spring 24 to achieve its purpose of providing a reliable pre-load on the ball end 16 for holding the connector housing assembly in place relative to the ball end 16. This may be referred to as a pre-clamping frictional engagement between the seats and the ball end to hold the seats in a selected position relative to the ball end. Thus the annular wave spring is configured to apply a concentrically uniform pre-clamping load to the assembled first and second seats 58 and 46 and the ball end 16.

### Methods of Manufacture of the Pedicle Screw Assembly

The pedicle screw assembly 10 is preferably pre-assembled by the manufacturer to include at least those components illustrated in the exploded view of FIG. 2. This assembly may be performed as follows.

The elongated screw 14 having the ball end 16 is placed through the open lower end 44 of lower housing part 20 and is engaged with lower seat 46. The spring 24 is placed about the upper smaller diameter portion 54 of sleeve 22, and the sleeve 22 is placed in engagement with the bore 30 and counterbore 32 of the upper housing part 18.

The upper and lower housing parts 18 and 20 are threadedly connected together by threads 48 and 50 thus assembling the elongated screw between the first and second seats, and pre-loading the seats toward each other with the annular wave spring 24 to provide a pre-clamping frictional engagement of the ball end 16 with the seats 58 and 46 so that the housing assembly 18, 20 containing the seats 58, 46 will remain in a selected position relative to the ball end 16 prior to applying a final clamping force to the seats.

The upper nut 26 is provided for later insertion within the upper housing part 18. The nut may or may not be pre-assembled with the upper housing part 18 by the manufacturer.

After the pedicle screw assemblies are put in place within the vertebra 12 as shown in FIG. 1, the elongated rods such as 28 are placed within the slots 40 of the upper housing parts 18. Then the nuts 26 are threadedly engaged with the internal threads of the upper housing to clamp the elongated rods 28 in place. This simultaneously clamps the ball end 16 between the upper and lower seats 58 and 46 to fix the position of the housing assembly relative to the ball end 16 and thus to fix the position of the elongated rods 28 relative to the pedicle screws 14.

### The Driver Assembly

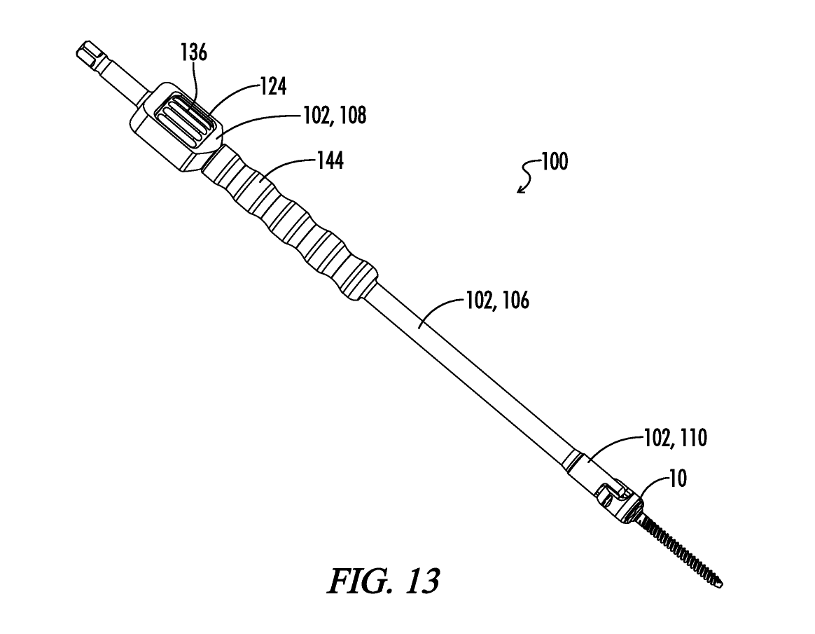

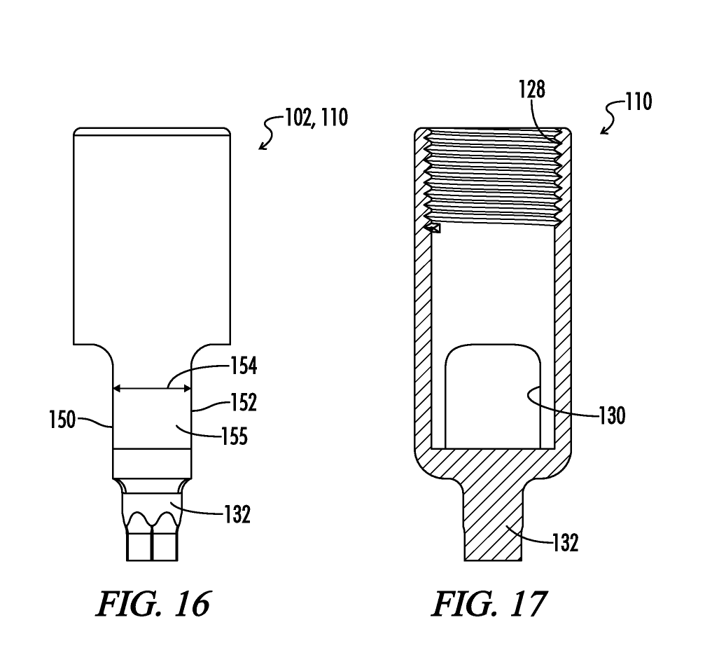

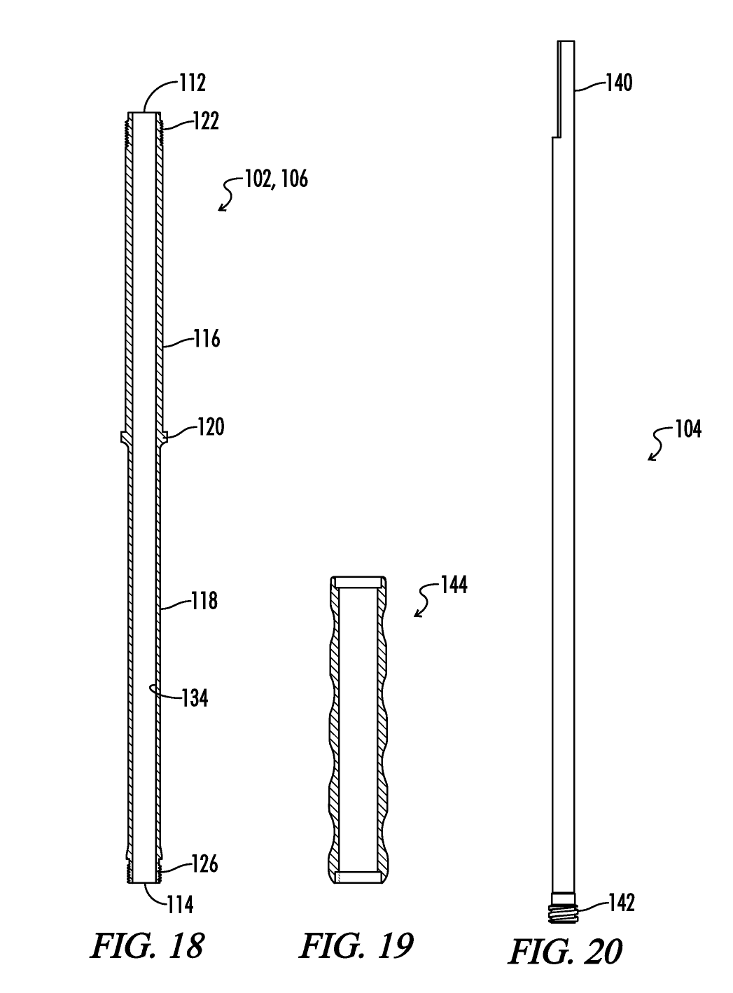

Referring now to FIGS. 13-24, a driver apparatus 100 for installing the pedicle screw assembly 10 is shown.

The driver apparatus 100 includes an elongated driver housing 102 and a release shaft 104 received in the driver housing 102.

The driver housing 102 is preferably a multi-part housing assembly including a tubular central driver housing portion 106, an upper driver housing portion 108, and a lower driver housing portion 110.

The central driver housing portion 106 includes upper and lower ends 112 and 114 which may also be referred to as proximal and distal ends 112 and 114.

The central driver housing portion 106 includes a larger diameter upper part 116 and a smaller diameter lower part 118 separated by an annular shoulder 120.

The upper driver housing portion 108 may also be referred to as an enlarged diameter knob housing portion 108, and is threadedly connected to the upper end 112 of central driver housing portion 106 by threads 122 and an internal thread (not shown) on the lower end of upper driver housing portion 108. The upper driver housing portion 108 has a first laterally open window 124 defined therethrough.

The lower driver housing portion 110, which may also be referred to as a distal housing portion 110, is attached to the lower end 114 of the central driver housing portion 106 by thread 126 on the central driver housing portion 106 and internal thread 128 on the lower driver housing portion 110. The lower driver housing portion 110 has a second laterally open window 130 defined therethrough.

The lower driver housing portion 110 has a screw drive head 132 defined thereon which has a multi-lobed shaped male driver configured to be received in a multi-lobed socket 17 defined in the upper end of the enlarged ball 16 of pedicle screw 14.

The central driver housing portion 106 has a longitudinal housing passage 134 defined therethrough which communicates with the upper laterally open window 124 and the lower laterally open window 130.

The release shaft 104 has a knob 136 attached to the upper end thereof. Knob 136 has a non-circular passage 138 which closely receives a non-circular upper portion 140 of the release shaft 104 so that the release shaft 104 and the knob 136 rotate together. As is best seen in FIG. 13, the knob 136 is received in and accessible through the upper laterally open window 124 defined in the upper housing portion 108.

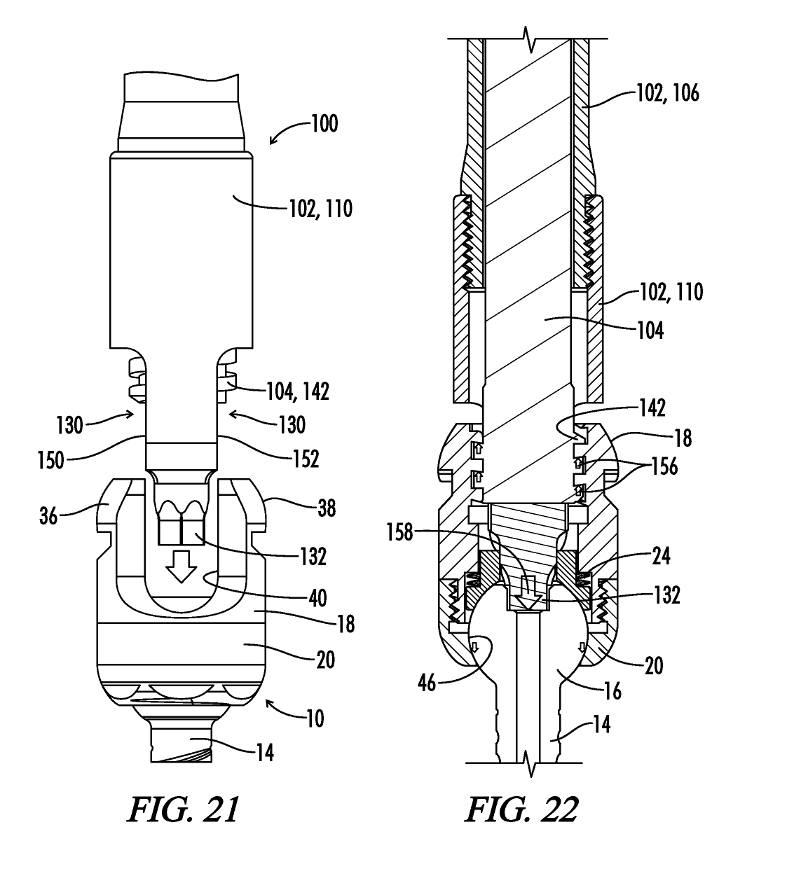

The release shaft 104 has a release thread 142 defined on a lower end portion of the shaft 104. The release thread 142 is received in and accessible through the lower laterally open window 130 as best seen in FIG. 21.

The release shaft 104 and knob 136 are rotatably received in the driver housing 102 so that the release thread 142 may be rotated by rotating the knob 136 while holding the driver housing 102 fixed.

An elongated spool handle 144 is best seen in FIG. 19 and is rotatably received about the larger diameter upper portion 116 of central driver housing portion 106.

The upper driver housing portion 108 has a drive shaft 146 extending upwardly therefrom, and the drive shaft 146 has an upper drive head 148 defined thereon which may be engaged by the surgeon with another drive tool to rotate the driver housing 102 while the surgeon steadies the driver housing 102 by grasping the spool handle 144. The driver housing 102 can rotate within the spool handle 144.

The lower driver housing portion 110 has first and second diametrically opposed parallel guide surfaces or guide surface portions 150 and 152 defined thereon on opposite sides of the lower laterally open window 130. The guide surface portions 150 and 152 are separated by a distance 154 which is less than the width 41 of slot 40 of the upper connector housing part 18 of the pedicle screw assembly 10. That portion of the lower driver housing 110 between the guide surfaces 150 and 152 may be referred to as a guide portion 155.

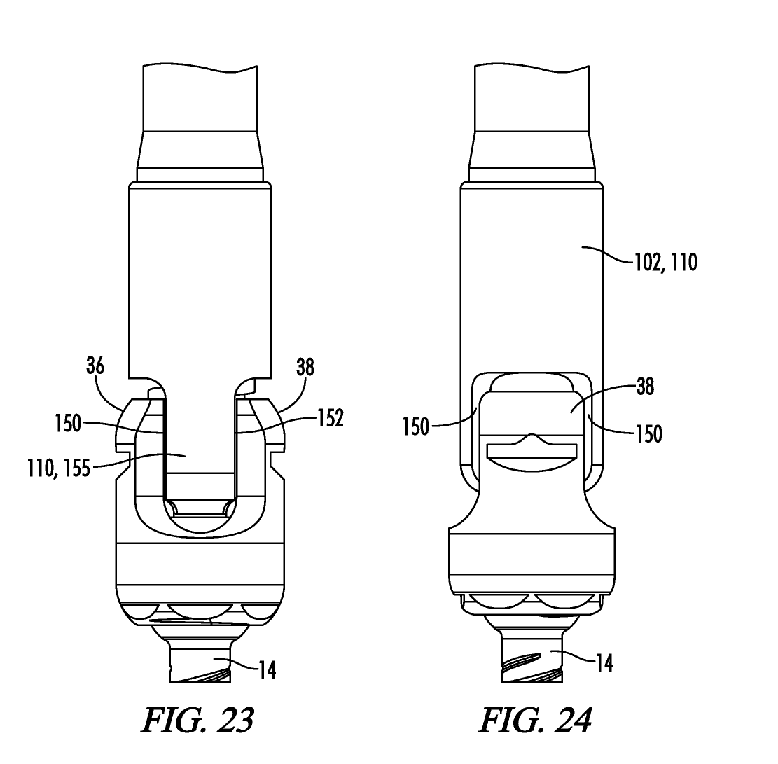

As seen in FIGS. 21 and 23, the guide surfaces 150 and 152 are configured to be closely received within the slot 40 of pedicle screw assembly 10 to hold the connector housing 18, 20 of the pedicle screw assembly 10 against rotation relative to the driver housing 102 when the release shaft 104 is rotated relative to the driver housing 102 to connect or disconnect the release thread 142 from the connector housing 18, 20 of the pedicle screw assembly 10.

As best seen in FIG. 22, the release thread 142 is configured to be threadedly engaged with the internal threads 68, 70 of the upper connector housing part 18 to connect or disconnect the driver apparatus 100 to or from the pedicle screw assembly 10 as is further described below.

### Manner of Operation

Referring now to FIGS. 21-24, the manner in which the driver apparatus 100 may be utilized to install the pedicle screw assembly 10 is further described.

After the spinal vertebrae 12 have been prepared in a known manner for placement of the pedicle screws 14, the driver apparatus 100 may be connected to one of the pedicle screw assemblies 10 in the manner illustrated in FIGS. 21-24. First, as shown in FIG. 21, guide portion 155 of the lower driver housing 110, is inserted into the slot 40 of the connector housing 18, 20 between the upward extending arms 36 and 38. The screw drive head 132 is placed into the drive socket 17 in the upper end of the enlarged ball end 16 as shown in FIG. 22. The release shaft 104 is rotated relative to the driver housing 102 to begin to make up the release thread 142 with the internal threads 68, 70 of the upper connector housing portion 18. As the threaded engagement 142, 68, 70 is further made up, the driver apparatus 100 pulls the connector housing assembly 18, 20 and the pedicle screw 14 upward relative to the driver housing 102 until the screw drive head 132 is fully engaged with the socket 17 as generally shown in FIG. 22.

It is noted that when the release thread 142 is fully made up with the internal threads 68, 70 of upper connector housing 18 as generally shown in FIGS. 22-24, the release thread 142 is substantially circumferentially enclosed by the longitudinally overlapping lower portion of the lower housing 110 and the upward extending arms 36 and 38 of the upper connector housing 18.

It is noted that by substantially circumferentially enclosing the release thread 142 the release thread 142 is generally protected from contact with the patient's body tissues as the pedicle screw assembly 10 is placed by the driver apparatus 100 into the patient's body.

With reference to FIG. 22 it is noted that when the release threads 142 are fully made up with the threads 68, 70 of upper connector housing part 18 to fully engage the screw drive head 132 in the socket 17 of ball end 16 of pedicle screw 14, a tension load is placed across the connector housing 18, 20. This tension load is generally represented by the arrows 156 indicating an upward directed force being applied against the threads 68, 70 and by the arrow 158 indicating a downward force being applied to the ball end 16 and thus to the lower seat 46 of connector housing 18, 20. This tension force, plus the engagement of the screw drive head 132 with the socket 17 of the ball end 16 of pedicle screw 14 aids in holding the pedicle screw assembly 10 and particularly the pedicle screw 14 and connector housing 18, 20 in axial alignment with the driver apparatus 100 as seen in FIG. 22 so as to resist misalignment of the pedicle screw 14 and the connector housing 18, 20 during the application of driving torque to the pedicle screw 14 by the driver apparatus 100.

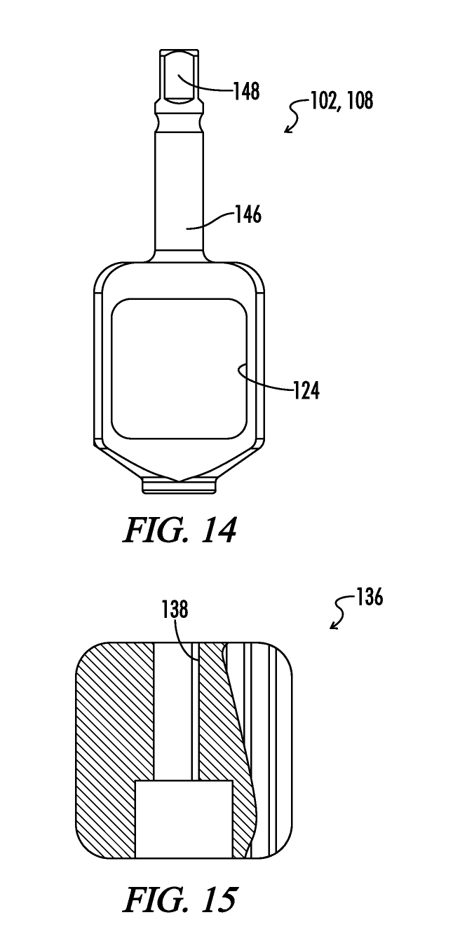

After the pedicle screw assembly 10 has been attached to the lower end of the driver apparatus 100 as generally shown in FIGS. 22-24, the pedicle screw 14 may be placed in the upper end of a prepared hole 13 of the spinal vertebra 12 and the surgeon may apply a driving torque to the pedicle screw 14 with the driver apparatus 100 thereby driving the pedicle screw 14 into position within the spinal vertebra 12 as generally shown in FIG. 1. This driving torque may be applied by rotating the driver housing 102 and particularly the screw drive head 132 defined on the lower end thereof while holding onto the spool handle 104. The driving torque may be applied to the driver housing 102 by placing a wrench or other tool on the upper drive head 148 seen in FIG. 14.

After the pedicle screw 14 has been driven into place within the spinal vertebra 12, the driver apparatus 100 may be released from the pedicle screw assembly 10 by unmaking the threaded engagement of the release thread 142 with the internal threads 68, 70 of the connector housing 18, 20. This is accomplished by holding the driver housing 102 fixed and rotating the release shaft and knob 136 counterclockwise relative to the driver housing 102 to unthread the release thread 102 from the connector housing 18, 20. The driver apparatus 100 is then withdrawn from the operating field.

At this point, the connector housing 18, 20 may be positioned by the surgeon in any desired rotational and angular relationship relative to the ball end 16, for example in positions like those shown in FIG. 1. The action of the annular wave spring 24 applies a pre-load between the upper and lower seats 58 and 46 across the ball end 16 so as to provide a frictional resistance to movement of the connector housing 18, 20 relative to the ball end 16.

Then, the elongated rods such as 28 may be placed in the slots 40 of a plurality of the pedicle screw assemblies 10, and then the elongated rods 28 may be clamped in place through the use of the upper nuts 26. The upper nuts 26 are placed in threaded engagement with the threads 68, 70 and are driven into place by another screwdriver (not shown) having a drive head which engages a socket 27 defined in the nut 26.

As the upper nut 26 is made up with the threads 68, 70 it bears down on the elongated rod 28 as seen in FIG. 1. The elongated rod 28 in turn bears down on the top of sleeve 22 which bears down via its seat 58 onto the spherical surface of ball end 16 which then bears down on the lower seat 46 to clamp the connector housing 18, 20 in place on the ball end 16 and to clamp the elongated rod 28 in place within the connector housing 18, 20 thus fixing the elongated rod 28 relative to the pedicle screw 14.

Thus it is seen that the apparatus and methods of the present invention readily achieve the ends and advantages mentioned as well as those inherent therein. While certain preferred embodiments of the invention have been illustrated and described for purposes of the present disclosure, numerous changes in the arrangement and construction of parts and steps may be made by those skilled in the art, which changes are encompassed within the scope and spirit of the present invention as defined by the appended claims.

What is claimed is:

1. A driver apparatus for a pedicle screw assembly, the pedicle screw assembly including a pedicle screw and a connector housing mounted on the pedicle screw, the driver apparatus comprising:
an elongated driver housing having a proximal housing end and a distal housing end, the distal housing end including a screw drive head configured for engaging the pedicle screw so that the pedicle screw can be rotated by rotating the driver housing, the driver housing having a first laterally open window located adjacent the proximal housing end and a second laterally open window located adjacent the distal housing end, the driver housing having a longitudinal housing passage defined therein communicating the first and second laterally open windows;
a release shaft extending through the longitudinal housing passage, the shaft having a knob attached to a proximal end portion of the shaft and the shaft having a release thread defined on a distal end portion of the shaft, the knob being received in and accessible through the first laterally open window and the release thread being received in and accessible through the second laterally open window, the release shaft being rotatable within the driver housing; and
wherein the driver housing includes a guide surface extending from the second laterally open window distally toward the screw drive head, the guide surface being configured to hold the connector housing of the pedicle screw assembly against rotation relative to the driver housing when the release shaft is rotated relative to the driver housing to disconnect the release thread from the connector housing of the pedicle screw assembly.

2. The apparatus of claim 1, further comprising:
an elongated spool handle rotatably received about the driver housing, the spool handle being located distally of the first laterally open window.

3. The apparatus of claim 1, wherein:
the first laterally open window is located in an enlarged diameter portion of the housing, and the first laterally open widow extends laterally through the housing and intersects the longitudinal housing passage; and
the knob protrudes through the first laterally open window on two diametrically opposed sides of the driver housing so that the knob can be grasped between the thumb and fingers of a human hand.

4. The apparatus of claim 1, wherein:
the driver housing comprises a multi-part housing assembly including a tubular central housing portion having a proximal end and a distal end, an enlarged diameter knob housing portion attached to the proximal end of the central housing portion and having the first laterally open window defined therein, and a distal housing portion attached to the distal end of the central housing portion and having the second laterally open window defined therein.

5. The apparatus of claim 1, wherein:
the guide surface comprises first and second diametrically opposed parallel guide surface portions on opposite sides of the lower laterally open window.

6. A pedicle screw installation system, comprising:
a pedicle screw assembly including a pedicle screw and a connector housing mounted on the pedicle screw, the pedicle screw including an enlarged ball end, the connector housing having a lower seat receiving the ball end with the screw extending downward through a lower opening of the connector housing, the connector housing having two upwardly extending arms having internal threads defined on the arms, the connector housing having a slot dividing the two arms; and
a driver apparatus, including:
an elongated driver housing including a screw drive head defined on a lower end of the driver housing, the screw drive head being configured for engaging the ball end of the pedicle screw so that the pedicle screw can be rotated by rotating the driver housing, the driver housing having an upper laterally open window and a lower laterally open window, the driver housing having a longitudinal housing passage defined therein communicating the upper and lower laterally open windows, the driver housing including first and second longitudinally extending guide surfaces on opposite sides of the lower laterally open window, the guide surfaces being configured to be received in the slot of the connector housing so that the connector housing may move longitudinally but not rotationally relative to the driver housing when the guide surfaces are received in the slot of the connector housing; and
a release shaft extending through the longitudinal housing passage, the shaft having a knob attached to the shaft, the knob being received in and accessible through the upper laterally open window, and the shaft having a release thread defined on the shaft and extending laterally through the lower laterally open window, the release thread being configured to be threadedly engaged with the internal threads of the two arms of the connector housing, the release shaft being rotatable within the driver housing to selectively engage or disengage the release threads from the internal threads of the arms of the connector housing to connect or disconnect the driver from the pedicle screw assembly.

7. The system of claim 6, wherein:
the driver apparatus and the pedicle screw assembly are configured such that when the release thread is fully engaged with the internal threads of the arms of the connector housing the screw drive head pushes downwardly against the pedicle screw and the release threads pull upwardly on the arms so that the connector housing is in tension and the pedicle screw and the connector housing are held in axial alignment with the driver apparatus so as to resist misalignment of the pedicle screw and the connector housing during the application of driving torque to the pedicle screw by the driver apparatus.

8. The system of claim 7, wherein:
the driver apparatus and the pedicle screw assembly are configured such that when the release thread is engaged with the internal threads of the arms of the connector housing and the guide surfaces are received in the slot of the connector housing, the longitudinally overlapping portions of the arms and the driver housing substantially enclose the release threads to shield the release threads from engagement with a patient's body tissue as the pedicle screw is inserted into the patient's body.

9. The system of claim 6, wherein:
the driver apparatus and the pedicle screw assembly are configured such that when the release thread is engaged with the internal threads of the arms of the connector housing and the guide surfaces are received in the slot of the connector housing, the longitudinally overlapping portions of the arms and the driver housing substantially enclose the release threads to shield the release threads from engagement with a patient's body tissue as the pedicle screw is inserted into the patient's body.

10. The system of claim 6, wherein:
the ball end of the pedicle screw has a drive socket formed therein; and
the screw drive head on the lower end of the driver housing is shaped as a male drive head complementary in shape to the drive socket for receipt in the drive socket.

11. A method of installing a pedicle screw assembly in a patient, the method comprising:
(a) providing a driver having a driver housing and having a release shaft rotatably received in the driver housing;
(b) providing the pedicle screw assembly including a pedicle screw and a connector housing, the connector housing having two upwardly extending arms with a slot between the arms;
(c) inserting a guide portion of a lower end of the driver housing into the slot of the connector housing;
(d) making up a threaded engagement of a release thread of the release shaft with internal threads of the upwardly extending arms of the connector housing;
(e) during step (d), advancing the guide portion of the lower end of the driver housing into the slot while preventing rotation of the connector housing relative to the driver housing, and forcing a drive head on a lower end of the driver housing into engagement with the pedicle screw; and
(f) substantially circumferentially enclosing the release threads within the arms of the connector housing and the guide portion of the driver housing.

12. The method of claim 11, further comprising:
placing a tension load across the connector housing between the pedicle screw and the internal threads of the arms, and thereby holding the pedicle screw and the connector housing in axial alignment with the driver so as to resist misalignment of the pedicle screw and the connector housing during the application of driving torque to the pedicle screw by the driver.

13. The method of claim 12, further comprising:
applying a driving torque to the pedicle screw with the driver and thereby driving the pedicle screw into the patient's body.

14. The method of claim 11, further comprising:
applying a driving torque to the pedicle screw with the driver and thereby driving the pedicle screw into the patient's body.

15. The method of claim 14, further comprising:
after driving the pedicle screw into the patient's body, unmaking the threaded engagement of the release thread of the release shaft with the internal threads of the upwardly extending arms of the connector housing to release the driver from the pedicle screw assembly.
